(19) United States
(12) Patent Application Publication
(10) Pub. No.: US 2002/0076807 A1
(43) Pub. Date: Jun. 20, 2002

(54) Cell observing method and the system thereof

(76) Inventors: Chi-Hung Lin, Taipei (TW); Jin-Wu Tsai, Chia-I City (TW); Yung-Hsing Yi, Taipei Hsien (TW)

Correspondence Address: BACON & THOMAS, PLLC, 4th Floor, 625 Slaters Lane, Alexandria, VA, 22314-1176, US

(21) Appl. No.: 09/739,703

(22) Filed: Dec. 20, 2000

(51) Int. Cl. C12M 1/34

(52) U.S. Cl. 435/288.7; 435/288.1; 435/288.3; 435/29; 359/398

(57) ABSTRACT

A method for observing the living cell has the steps of erecting a substrate for the living cell; cultivating the living cell on the erected substrate; and observing the living cell with an optical microscope. The system used to incorporate the method has a microscope; and a culture dish having a body with an opening defined in a bottom of the body and a coverslip provided to cover the opening.

### BACKGROUND OF THE INVENTION

1. Field of the Invention

The present invention relates to a cell observing method and more particularly to a cell observing method having the cell to be observed cultivated on a vertical wall so as to facilitate the real-time observation to the living cell with an optical microscope.

2. Description to Prior Art

Currently, the observation to the living cell is to observe the cell directly on top of the cell that is cultivated on a coverslip or a slide of a culture dish. Normally, a substrate is provided on the slide to enable the observed cell to grow. During the growth of the observed cell, some of the extracellular matrix receptor will adhere to the substrate and trigger a morphological changes. This kind of changes will cause different Morphology to the cell or the tissue type of the cell when observed from different sections.

The conventional cell observing technique is applied directly on top of the cell so that it is very difficult to have the accurate understanding and observation to the “Depth” of the cell, which limits the understanding to the structure and the activities of the living cell of many important creatures. Early resolution to this problem is to use the continuous sectioning to the observed cell and then reconstruct the structure of the cell, which is very time consuming and above all, this continuous sectioning technique can only be applied to the “Fixed” dead cell. This can not be used to observe the reaction or process of movement of the living cell, thus the research to the living cell improves quite slowly.

In order to solve the problem, in 1960s, a magazine called “NATURE” released a report using a material similar to a prism to reflect the light to enable the researcher to see the side view of the living cell from the bottom. However, the resolution of this technique is not high and is very troublesome so that it is quite difficult to be popularly applied in general microscopes. Because of the difficulties, later research in the cell did not pay too much attention to the observation technique until the introduction of the laser scanning confocal microscope. The introduction of the laser scanning confocal microscope triggers a revolution in the 3D observation to the living cell, which requires no difficult sectioning to achieve the goal of observing the living cell from its side view.

However, this kind of method adopts dot scanning to the living cell to reconstruct the structure of the cell, which requires an overall 3D scanning to the cell so as to construct the entire structure of the cell. This dot scanning, even if it scans only one plan face, requires enormous amount of time to accomplish the work, which reduces the effectiveness and value of the method in the cell structure and cell movement research. Furthermore, if a high resolution to the scanned cell is requires, the researcher must slow down the scanning speed, which lengthens the overall time of the research. Besides, the laser scanning confocal microscope is expensive, thus a general researcher can not afford such a device. What is more is that it takes more than a month to train an operator to be familiar with the laser scanning confocal microscope. All these reasons causes the observation to the living cell stalled.

Thus, it is known that none of the existing techniques in observing the living cell can:

1. observe the living cell or tissue;

2. achieve the optical and resolution requirement;

3. observe in real time; and

4. be applied in various microscopes, such as bright field, dark field, phase contrast, differential interference contrast (DIC) and fluorescence microscopes.

It is therefore concluded that the conventional technique used in observing the living cell depends entirely on the laser scanning confocal microscope, which processes the structure construction by dot scanning the living cell or the tissue. That is, an image of a cell will have to undertake a thorough 3D scanning before the structure is constructed, with which even a plan surface is scanned, it still a waste of time. Because of the resolution requirement, the time required to complete the thorough scanning can not be shortened. Furthermore, it takes more than a month to train a qualified researcher to be familiar with the laser scanning confocal microscope, which is too time consuming and also the cost for the laser scanning confocal microscope is too expensive. All these reasons limit the improvement in the observation to the living cell.

Accordingly, it is an objective of the invention to provide an improved method to obviate and mitigate the aforementioned problems.

### SUMMARY OF THE INVENTION

The primary objective of the invention is to provide an improved method to observe the living cell. This method cultivates the living cell on a vertical wall, so that with the normal microscope, even a general laboratory can have the complete structure of the living cell and proceed the observation to the movement of the living cell.

Another objective of the invention is to provide the system used to complete the observation to the living cell. The system includes a semi-permeable or porous material applied on a vertical wall to proceed electrical, magnetic or chemical treatment to the living cell so as to facilitate the observation to the living cell.

Still another objective of the invention is to provide the system used to complete the observation to the living cell. The system uses a semiconductor applied on a vertical wall to proceed electrical, magnetic or chemical treatment to the living cell so as to facilitate the observation to the living cell.

Other objects, advantages and novel features of the invention will become more apparent from the following detailed description when taken in conjunction with the accompanying drawings.

### BRIEF DESCRIPTION OF THE DRAWINGS

FIG. 1 is a schematic view of the system of the invention;

FIG. 2 is a perspective view of the first preferred embodiment of the culture dish of the invention;

FIG. 3 is a cross sectional view of the culture dish of FIG. 1 by taking the line 3-3;

FIG. 4 is a perspective view of the second preferred embodiment of the culture dish of the invention;

FIG. 5 is a cross sectional view of the culture dish of FIG. 4;

FIG. 6 is a cross sectional view of the third preferred embodiment of the culture dish in accordance with the present invention;

FIG. 7 is a schematic view showing the method of the invention;

FIG. 8 is a schematic view showing another way of completing the method of the invention;

FIG. 9 is a cross sectional view of the fourth preferred embodiment of he culture dish in accordance with the present invention; and

FIG. 10 is a cross sectional view of the fifth preferred embodiment of the culture dish in accordance with the present invention.

### DETAILED DESCRIPTION OF THE PREFERRED EMBODIMENT

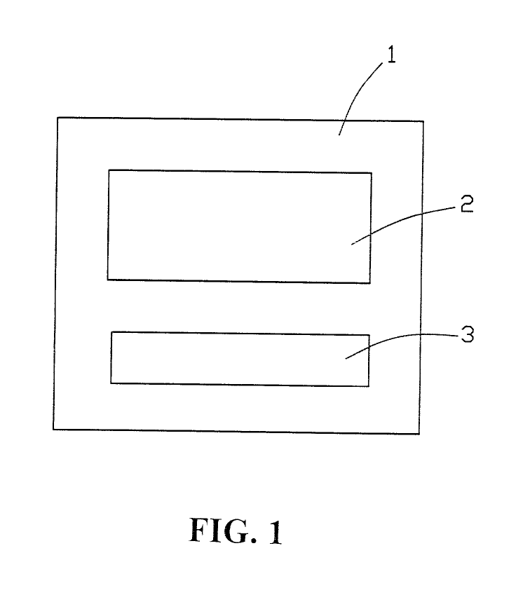

With reference to FIG. 1, the system used to observe the living cell includes at least a microscope 2 and a cell culture dish (or a cell culture flask) 3. The microscope 2 may be a bright field, dark field, phase contrast, differential interference contrast (DIC) and fluorescence microscope. The culture dish 3 may also be a culture flask.

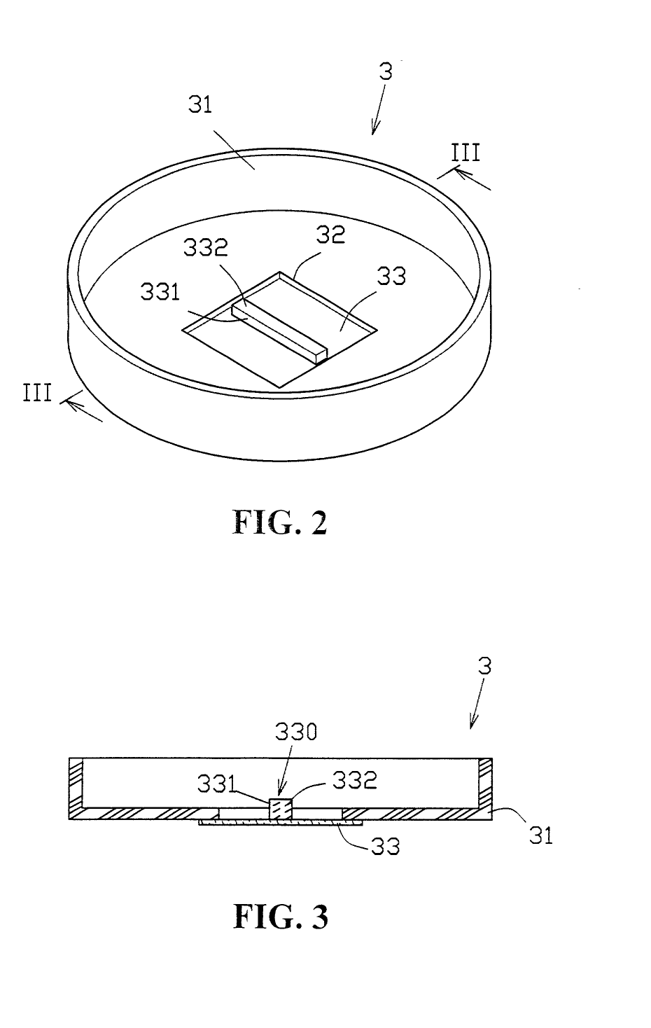

With reference to FIGS. 2 and 3, the culture dish 3 has a body 31 with an opening 32 defined in a bottom of the body 31 and a coverslip 33 provided to cover the opening 32 and having a thickness of 0.17 mm. The coverslip 33 has an observing cube 330 securely engaged with the coverslip 33 and having a side face 331 and an observing face 332 vertical to the side face 331. Because the working distance of the objective of the microscope 2 is very short, which limits the thickness of the coverslip 33. The preferred thickness of the coverslip 33 is maintained at 0.17 mm to support the observing cube 330. In order to have high resolution, the smoothness of the side face 331 and the observing face 332 and the angle therebetween should be highly demanded. Furthermore, the material used to make the observing cube 330 should choose the one with lower expansion coefficient.

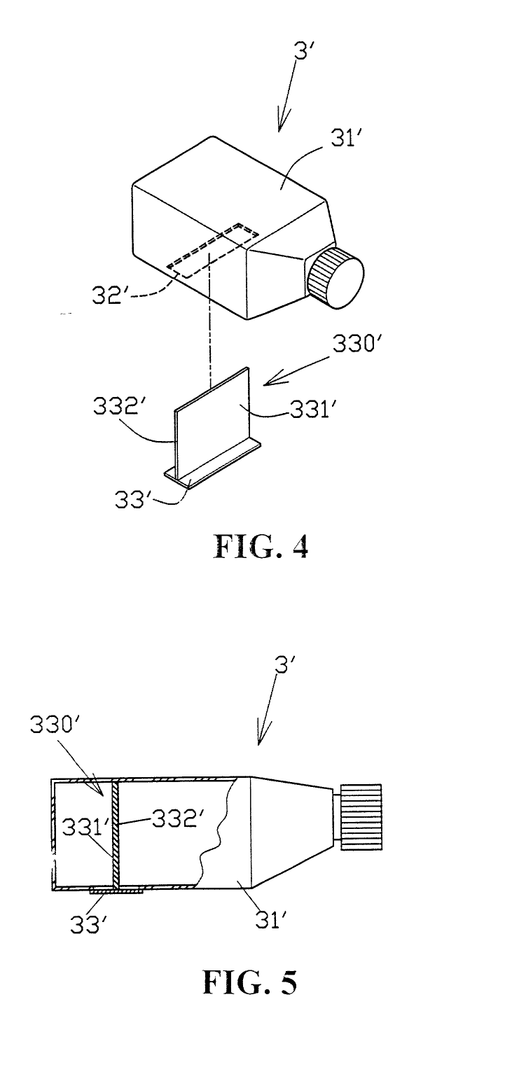

With reference to FIGS. 4 and 5, when the culture dish 3 is changed to a culture flask 3′, due to the roughness and the thickness of the culture flask 3′, the culture flask 3′ normally is incorporated with a microscope of low magnification. In order to use the culture flask 3′ in the present invention, the culture flask 3′ has an opening 32′ defined in a bottom of the culture flask 3′, a coverslip 33′ provided to cover the opening 32′ and an observing cube 330′ with a side face 331 ′ and an observing face 332′ vertical to the side face 331′. Because the working distance of the objective of the microscope 2 is very short, which limits the thickness of the coverslip 33′. The preferred thickness of the coverslip 33′ is maintained at 0.17 mm to support the observing cube 330′. In order to have high resolution, the smoothness of the side face 33 1′ and the observing face 332′ and the angle therebetween should be highly demanded. Furthermore, the material used to make the observing cube 330′ should choose the one with lower expansion coefficient.

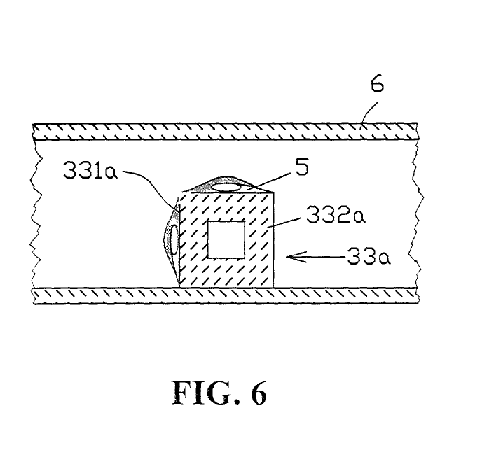

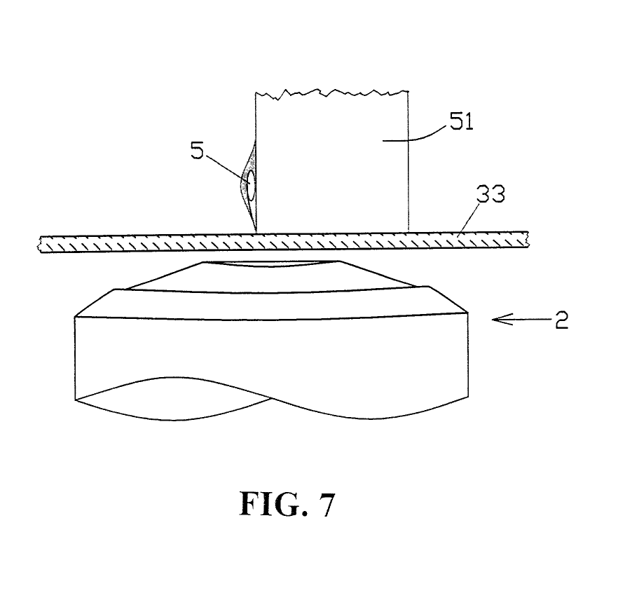

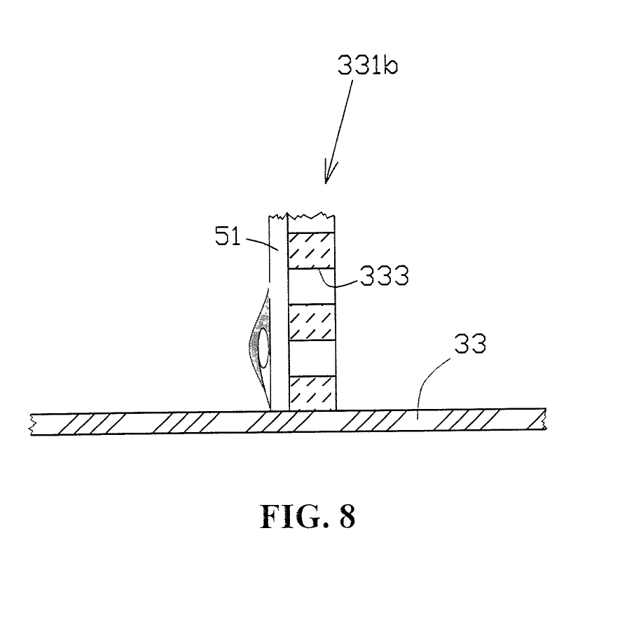

With reference to FIG. 6, the culture disk 3 is now changed to a glass-made cube 33a having a side face 331a and an observing face 332a. the cube 33a is placed in a chamber 6 filled with water. As shown in FIGS. 7 and 8, the principle used in this method is to erect the substrate 51 for the cell 5 to therefore form a side face, such that a researcher is able to observe the cell 5 with an optical microscope from the top or the bottom of the cell 5. That is, to cultivate the cell 5 on a vertical face 331 made of a porous or semi-permeable material to proceed electrical, magnetic or chemical treatment to the living cell so as to facilitate the observation to the living cell. The multiple apertures 333 are defined in the vertical face 331 to form a porous feature.

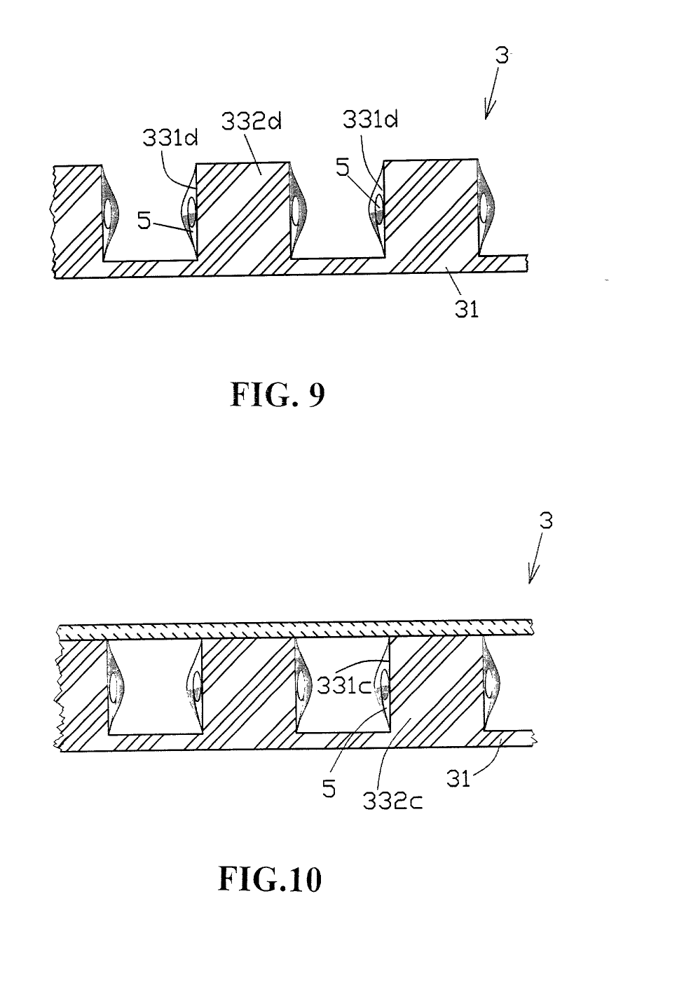

With reference to FIG. 9, a continuous side faces 331d are formed on the body 31 by etching so as that multiple observation faces 332d are thus formed.

With reference to FIG. 10, the body 3 is a base of another material such as a silicon or a conductor and multiple side faces 331c and observation faces 332c are formed by etching.

While processing the method in accordance with the invention, there are points should be particularly noted:

1. thickness;

Due to the short working distance of the objective of a microscope, the thickness of the coverslit should be kept in minimum. After experiments, the thickness of the coverslit is preferably made to be 0.17 mm. Furthermore, if the coverslit is made integrally with the body, a better performance is achieved.

2. smoothness

Because the objective has short working distance in the high resolution microscope, the smoothness of the coverslit should take particular caution and the material for the coverslit should choose the material with low expansion coefficient.

According to the above description, the present invention has the following advantages:

1. There has no need to proceed 3D scanning to know the structure of a observed cell, such that the overall time spent should be kept in minimum. Due to the direct observation to the living cell, a real-time observation is achieved.

2. Using the porous or semi-permeable material to form the side face that is used to cultivate the cell enables the researcher o break through the limitation to the resolution of the optical microscope.

It is to be understood, however, that even though numerous characteristics and advantages of the present invention have been set forth in the foregoing description, together with details of the structure and function of the invention, the disclosure is illustrative only, and changes may be made in detail, especially in matters of shape, size, and arrangement of parts within the principles of the invention to the full extent indicated by the broad general meaning of the terms in which the appended claims are expressed.

What is claimed is:

1. A cell observation system comprising:
a microscope; and
a culture dish having a body with an opening defined in a bottom of the body and a coverslip provided to cover the opening.

2. The system as claimed in claim 1, wherein a side face adapted to cultivate the cell is formed on the coverslit and is made of a semi-permeable material.

3. The system as claimed in claim 1, wherein a side face adapted to cultivate the cell is formed on the coverslit and is made of a porous material.

4. The system as claimed in claim 1, the microscope is a bright field microscope.

5. The system as claimed in claim 2, the microscope is a bright field microscope.

6. The system as claimed in claim 3, the microscope is a bright field microscope.

7. The system as claimed in claim 1, the microscope is a dark field microscope.

8. The system as claimed in claim 2, the microscope is a dark field microscope.

9. The system as claimed in claim 3, the microscope is a dark field microscope.

10. The system as claimed in claim 1, the microscope is a phase contrast microscope.

11. The system as claimed in claim 2, the microscope is a phase contrast microscope.

12. The system as claimed in claim 3, the microscope is a phase contrast microscope.

13. The system as claimed in claim 1, the microscope is a differential interference contrast microscope.

14. The system as claimed in claim 2, the microscope is a differential interference contrast microscope.

15. The system as claimed in claim 3, the microscope is a differential interference contrast microscope.

16. The system as claimed in claim 1, the microscope is a fluorescent microscope.

17. The system as claimed in claim 2, the microscope is a fluorescent microscope.

18. The system as claimed in claim 3, the microscope is a fluorescent microscope.

19 The system as claimed in claim 1, wherein a material of low expansion coefficient is chosen to make the culture dish.

20. A method for observing the living cell comprising the steps of:
erecting a substrate for the living cell;
cultivating the living cell on the erected substrate; and
observing the living cell with an optical microscope.

21. The method as claimed in claim 20, the substrate is in a culture dish.

22. The method as claimed in claim 20, the substrate is in a culture flask.

23. The method as claimed in claim 21, the culture dish is etched to form a continuous side faces and observation faces.

24. The method as claimed in claim 20, a step of processing electrical stimulation is added to the living cell before the observing step.

25. The method as claimed in claim 20, a step of processing chemical treatment is added to the living cell before the observing step.

26. The method as claimed in claim 20, a step of processing magnetic stimulation is added to the living cell before the observing step.

27. The method as claimed in claim 23, a semiconductor is added to the side faces.

28. The method as claimed in claim 27, a step of processing electrical stimulation is added to the living cell before the observing step.

29. The method as claimed in claim 27, a step of processing chemical treatment is added to the living cell before the observing step.

30. The method as claimed in claim 27, a step of processing magnetic stimulation is added to the living cell before the observing step.
